(19) United States
(12) Patent Application Publication
(10) Pub. No.: US 2023/0079796 A1
(43) Pub. Date: Mar. 16, 2023

(54) TISSUE CONTACTING DEVICE FOR ORAL AND OTHER MEDICAL PROCEDURES

(71) Applicant: ARMOR DENTAL, CORP., Hingham, MA (US)

(72) Inventors: Cherie Le Penske, Hingham, MA (US); Andrew W. Santin, Beverly, MA (US); Gregory E. Healey, Nyack, NY (US)

(21) Appl. No.: 17/942,342

(22) Filed: Sep. 12, 2022

Related U.S. Application Data
(60) Provisional application No. 63/242,651, filed on Sep. 10, 2021.

(51) Int. Cl. A46B 5/00; A46B 5/02

(52) U.S. Cl. CPC A46B 5/0095; A46B 5/026; A46B 2200/1066

(57) ABSTRACT

A system for laser-based medical use and laser-based oral surgery. The system includes an elongate handle having a proximal end and a distal end; a (e.g., disposable, selectively removable) functional element disposed at the distal end of the elongate handle; and an integral, soft coating material formed about, so as to cover, the functional element and the distal end of the elongate handle. In some implementations, the elongate handle includes a rigid (e.g., polycarbonate) portion and a soft (e.g., thermoplastic elastomer) portion for improving a user's grip of the elongate handle.

### CROSS-REFERENCE TO RELATED APPLICATION

The present application claims priority to U.S. Provisional Patent Application No. 63/242,651 filed Sep. 10, 2021, which is hereby incorporated by reference herein in its entirety.

### TECHNICAL FIELD OF THE INVENTION

The invention relates generally to functional elements and systems for laser-based medical use, laser-based oral surgery, lingual assessments/diagnoses, airway assessments/diagnoses, and so forth. More particularly, the invention relates to functional elements and systems for laser-based medical use and laser-based oral surgery that include a substantially planar upper surface having an arcuate perimeter, a substantially planar lower surface having an arcuate perimeter, and a convex sidewall extending from the upper surface to the lower surface and/or are more laser-compliant than conventional functional elements. Although the invention will be described in the context of laser-based medical use and laser-based oral surgery, those of ordinary skill in the art can appreciate that functional elements and systems may also be used for intubation and gynecological, as well as other medical and veterinary, uses.

### BACKGROUND OF THE INVENTION

As laser-based medical procedures, including, without limitation, oral surgery and veterinary surgery, replace more traditional surgery procedures, functional elements or systems that are more compatible with the laser functional elements are warranted. For example, to prevent injury or damage to the tongue and soft tissue in the mouth of a patient during a (e.g., lingual) frenulotomy, means for stabilizing and properly positioning the patient's tongue are required. Conventional techniques for stabilizing the patient's tongue include using the surgeon's finger(s) or an (e.g., tongue extraction) instrument to support the patient's tongue as the frenulum is divided (e.g., using a laser). Such functional elements designed to lift the tongue are typically manufactured from stainless steel and include a grooved or slotted portion.

Heretofore, parents whose infant or child has undergone a “tongued tied” procedure are inclined to use their digits to exercise (i.e., lift) the infant's/child's tongue after the (e.g., lingual) frenulotomy surgery to prevent the tongue from reattaching itself.

Conventional functional elements also interact poorly with laser energy. Many conventional functional element can reflect the laser energy such that it could impinge upon unintended tissue regions, which can be a safety hazard to the patient or operator. Other conventional functional elements smoke or become excessively hot when interacting with laser energy.

### SUMMARY OF THE INVENTION

In a first aspect, the present invention relates to a system for laser-based medical use and laser-based oral surgery. In some embodiments, the system includes an elongate handle having a proximal end and a distal end; a (e.g., disposable, selectively removable) functional element disposed at the distal end of the elongate handle; and an integral, soft coating material formed about, so as to cover, the functional element and the distal end of the elongate handle. In some implementations, the elongate handle includes a rigid (e.g., polycarbonate) portion and a soft (e.g., thermoplastic elastomer) portion for improving a user's grip of the elongate handle. The functional element may include a mirror, a tissue retraction element, a laser blocking element, a brush, a hook, a block, and/or a buccal tip. The integral, soft covering may be manufactured of a soft polymer selected from the group consisting of a thermoplastic elastomer, a thermoplastic elastomer having a durometer value between 10 and 25 Shore A, or a thermoplastic elastomer having a durometer value between 30 and 40 Shore A.

In some applications, the functional element forms a tight, interference fit with the distal end of the elongate handle. Moreover, the distal end of the elongate handle may include a quarter-turn twist on element and/or a ball detent for attaching the functional element to the distal end of the elongate handle.

In some variations, the system further may also include a (e.g., disposable) second functional element that is selectively attachable to and removably from the proximal end of the elongate handle. The second functional element may include a mirror, a tissue retraction element, a laser blocking element, a brush, a hook, a block, and/or a buccal tip. Optionally, the proximal end of the elongate handle may include an indent feature that is structured and arranged to mate with a projection feature formed in the (e.g., disposable) second functional element. Alternatively, the proximal end of the elongate handle may include a projection feature that is structured and arranged to mate with an indent feature formed into the (e.g., disposable) second functional element.

In some embodiments, an element, pin, and/or rod may be disposed in a neck portion of the distal end of the elongate handle. The element, pin, and/or rod may be selected from the group consisting of stainless steel, aluminum 1100, fully annealed copper C1100, and stainless steel 316. Advantageously, the element, pin, and/or rod is pliable so that neck portion is formable to provide a gooseneck appearance (function).

When the functional element is a mirror, the mirror may be disposed within a substrate manufactured from at least one of plastic, a thermoplastic elastomer, and combinations thereof. Advantageously, the integral, soft coating material seals about a peripheral surface of the mirror and/or the forms a bumper about a peripheral surface of the mirror.

In a second aspect, the present invention relates to an oral hygiene kit that includes the system and at least one oral hygiene functional element selected from the group consisting of a tooth brush, a flossing device, and a gingiva stimulating device.

In a third aspect, the present invention relates to a system for laser-based medical use and laser-based oral surgery. In some embodiments, the system includes an elongate handle having a proximal end and a distal end and a functional element operatively coupled to the distal end of the elongate handle. In some implementations, the functional element includes: a substantially planar upper surface having an arcuate perimeter, a substantially planar lower surface having an arcuate perimeter, a convex sidewall extending from the upper surface to the lower surface, and an indent (e.g., a slot) formed at a distal end of the functional element.

In a fourth aspect, the present invention relates to a system for laser-based medical use and laser-based oral surgery. In some embodiments, the system includes an arcuate functional element that, in some implementations, includes: a substantially planar upper surface having an arcuate perimeter; a substantially planar lower surface having an arcuate perimeter; a convex sidewall extending from the upper surface to the lower surface; and a slot formed at the distal end of the functional element, wherein opposing walls of the slot are chamfered.

In a fifth aspect, the present invention relates to a system for laser-based medical use and laser-based oral surgery. In some embodiments, the system includes an arcuate functional element that, in some implementations, includes: a substantially planar upper surface having an arcuate perimeter, a substantially planar lower surface having an arcuate perimeter; a convex sidewall extending from the upper surface to the lower surface, and an indent (e.g., a slot) formed at a distal end of the functional element.

In a sixth aspect, the present invention relates to a functional element for use with oral or medical surgery that is more laser compliant than conventional functional elements. As used herein, laser compliant means that the functional element exhibits a safer and/or favorable responses to interaction with a laser than conventional functional elements. For example, the functional element may exhibit a lower increase in temperature, lower reflectivity of laser radiation, and/or less smoking or charring when interacting with laser energy than conventional functional elements. In some embodiments, the functional element is particularly configured to have superior performance/interaction with a particular type of laser (e.g., Er: YAG, diode-based, or CO₂) and/or to have superior performance/interaction with a particular laser wavelength (e.g., in a range from 2-4 μm, in a range from 9-11 μm).

In some embodiments, superior laser compatibility can be a result of the type, color, and/or translucence of the material from which the laser interacting portion of the functional element is formed. Through testing, Applicant has discovered that a solid white TPE component can interact with a diode laser with no adverse effect (e.g., no sparking, smoking, etc.). Applicant also discovered that a cloudy/clear TPE tip can interact with an Er: YAG laser with no adverse effect (e.g., no sparking, smoking, etc.). In some embodiments, the use or water (or other coolant) in conjunction with delivery of the laser energy can affect the types of materials that are laser compliant. For example, Applicant discovered that a cloudy/clear TPE material will spark when interacting with a CO₂ laser without water, but does not spark or have other adverse effect when interacting with a CO₂ laser with water. Applicant has discovered that a cloudy/clear TPE material can interact with an Er: YAG laser (e.g., manufactured by Fotona™) or a diode-based laser (e.g., manufactured by Biolase™) without generating any smoke or spark, regardless of whether water is present. Furthermore, Applicant has discovered that a cloudy/clear TPE material can interact with a CO₂ laser (e.g., manufactured by Convergent Dental™) without generating any smoke or spark, if water is present.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention. In the following description, various embodiments of the present invention are described with reference to the following drawings, in which:

FIG. 1A shows a side, elevation view of a system for laser-based medical use and laser-based oral surgery, in accordance with some embodiments of the present invention;

FIG. 1B shows a top, plan view of the system of FIG. 1A, in accordance with some embodiments of the present invention;

FIG. 1C shows a top perspective view of a first embodiment of a distal end for the system of FIG. 1A, in accordance with some embodiments of the present invention;

FIG. 1D shows a top, plan view of the distal end of FIG. 1C, in accordance with some embodiments of the present invention;

FIG. 1E shows a top perspective view of a chamfered, second embodiment of a distal end for the system of FIG. 1A, in accordance with some embodiments of the present invention;

FIG. 1F shows a distal end view of the chamfered, second embodiment of the distal end of FIG. 1E, in accordance with some embodiments of the present invention;

FIG. 1G shows a detail of the chamfered opposing walls of the second embodiment of the distal end of FIG. 1E, in accordance with some embodiments of the present invention;

FIG. 1H shows a top, plan view of the distal end of the chamfered, second embodiment of the distal end of FIG. 1E, in accordance with some embodiments of the present invention;

FIG. 1I shows a distal end view of a chamfered, third embodiment of the distal end for the system of FIG. 1A, in accordance with some embodiments of the present invention;

FIG. 1J shows top, plan view of the distal end of the chamfered, third embodiment of the distal end of FIG. 1I, in accordance with some embodiments of the present invention;

FIG. 1K shows a detail of the chamfered opposing walls of the third embodiment of the distal end of FIG. 1I, in accordance with some embodiments of the present invention;

FIG. 2A shows a side, elevation view of another system for laser-based medical use and laser-based oral surgery, in accordance with some embodiments of the present invention;

FIG. 2B shows a top, plan view of the system of FIG. 2A, in accordance with some embodiments of the present invention;

FIG. 2C shows a top perspective view of an embodiment of a distal end for the system of FIG. 2A, in accordance with some embodiments of the present invention;

FIG. 2D shows a distal end view of the system of FIG. 2A, in accordance with some embodiments of the present invention;

FIG. 2E shows a top, plan view of the distal end of FIG. 2C, in accordance with some embodiments of the present invention;

FIG. 3A shows a side, elevation view of yet another system for laser-based medical use and laser-based oral surgery, in accordance with some embodiments of the present invention;

FIG. 3B shows a top, plan view of the system of FIG. 3A, in accordance with some embodiments of the present invention;

FIG. 3C shows a top perspective view of an embodiment of a distal end for the system of FIG. 3A, in accordance with some embodiments of the present invention;

FIG. 3D shows a distal end view of the system of FIG. 3A, in accordance with some embodiments of the present invention;

FIG. 3E shows a top, plan view of the distal end of FIG. 3C, in accordance with some embodiments of the present invention;

FIG. 4A shows a cross-sectional view of a universal handle and disposable functional element connectable using a snap-on with a ball detent, in accordance with some embodiments of the present invention;

FIG. 4B shows a perspective view of the snap-on with a ball detent of FIG. 4A, in accordance with some embodiments of the present invention;

FIG. 4C shows a further perspective view of the snap-on with a ball detent of FIG. 4B, in accordance with some embodiments of the present invention;

FIG. 4D shows a cross-section view of a universal handle and disposable functional element connectable using a quarter-turn twist lock detent, in accordance with some embodiments of the present invention;

FIG. 4E shows a perspective view of the quarter-turn twist lock detent of FIG. 4D, in accordance with some embodiments of the present invention;

FIG. 4F shows a further perspective view of the quarter-turn twist lock detent of FIG. 4E, in accordance with some embodiments of the present invention;

FIG. 5A shows a top (plan) view of a buccal tip functional element, in accordance with some embodiments of the present invention;

FIG. 5B shows a side view of the buccal tip functional element of FIG. 5A, in accordance with some embodiments of the present invention;

FIG. 5C shows a top perspective view of the buccal tip functional element of FIG. 5A, in accordance with some embodiments of the present invention;

FIG. 5D shows a cross-sectional view of the buccal tip functional element of FIG. 5B, in accordance with some embodiments of the present invention;

FIG. 5E shows a plan (top) view of the buccal tip functional element of FIG. 5A attached to an end of an elongate handle, in accordance with some embodiments of the present invention;

FIG. 6A shows a side view of a soft retraction functional element with an integral soft coating, in accordance with some embodiments of the present invention;

FIG. 6B shows a top (plan) view of the soft retraction functional element of FIG. 6A, in accordance with some embodiments of the present invention;

FIG. 6C shows a cross-sectional view of the soft retraction functional element of FIG. 6A, in accordance with some embodiments of the present invention;

FIG. 6D shows a top perspective view of the soft retraction functional element of FIG. 6A, in accordance with some embodiments of the present invention;

FIG. 7A shows a side view of a soft mirror functional element with an integral soft coating, in accordance with some embodiments of the present invention;

FIG. 7B shows a top perspective view of the soft mirror functional element of FIG. 7A, in accordance with some embodiments of the present invention;

FIG. 7C shows a cross-sectional view of the soft mirror functional element of FIG. 7A, in accordance with some embodiments of the present invention; and

FIG. 7D shows a top perspective view of the soft mirror functional element of FIG. 7A, in accordance with some embodiments of the present invention.

### DETAILED DESCRIPTION

The terms and expressions employed herein are used as terms and expressions of description and not of limitation, and there is no intention, in the use of such terms and expressions, of excluding any equivalents of the features shown and described or portions thereof. In addition, having described certain embodiments of the invention, it will be apparent to those of ordinary skill in the art that other embodiments incorporating the concepts disclosed herein may be used without departing from the spirit and scope of the invention. The features and functions of the various embodiments may be arranged in various combinations and permutations, and all are considered to be within the scope of the disclosed invention. Unless otherwise necessitated, recited steps in the various methods may be performed in any order and certain steps may be performed substantially simultaneously. Accordingly, the described embodiments are to be considered in all respects as only illustrative and not restrictive. Furthermore, the configurations, materials, and dimensions described herein are intended as illustrative and in no way limiting. Similarly, although physical explanations have been provided for explanatory purposes, there is no intent to be bound by any particular theory or mechanism, or to limit the claims in accordance therewith.

Tongue Controlling Functional Elements

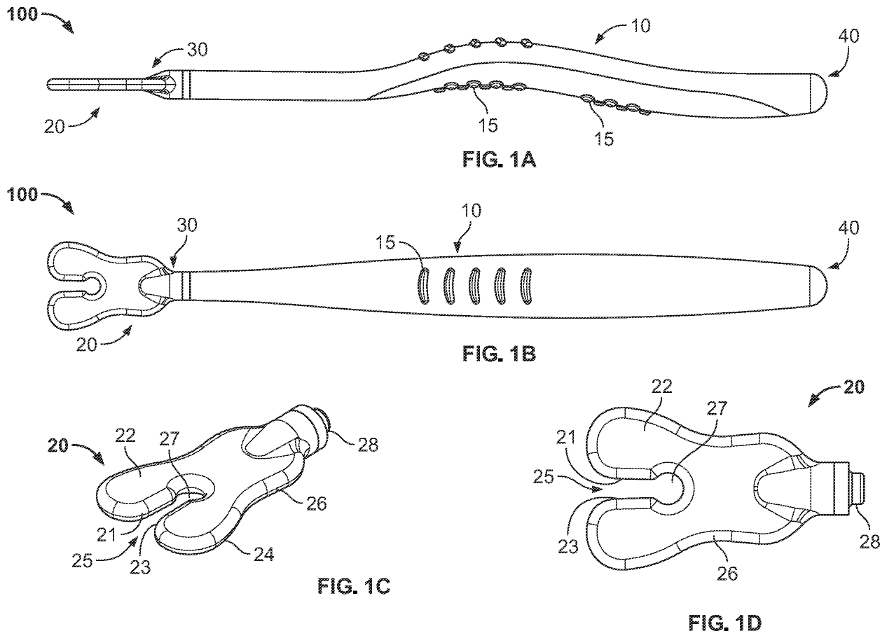

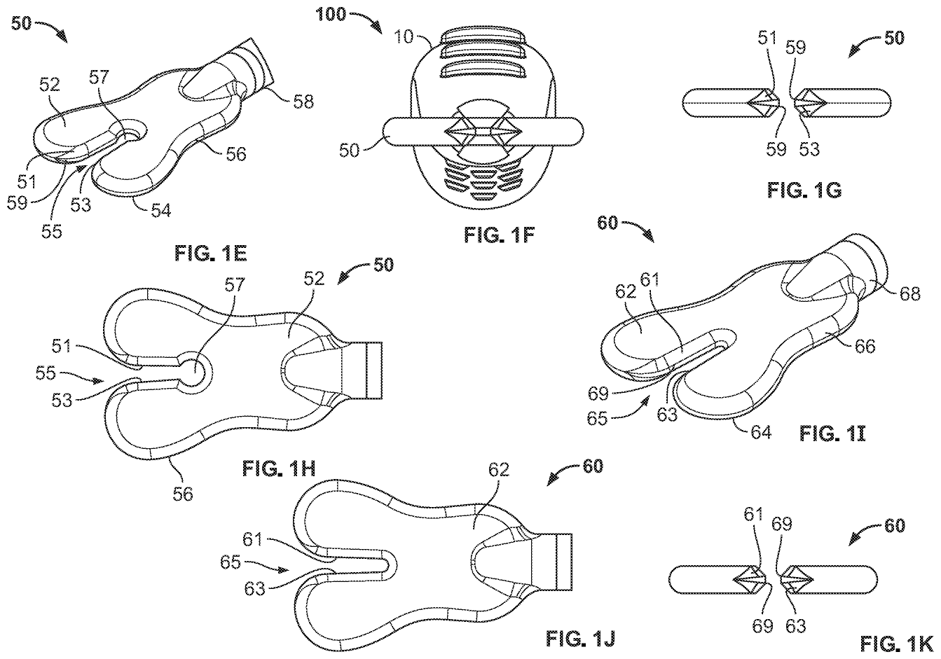

Referring to FIGS. 1A and 1B, a first embodiment of a system for (e.g., laser-based) medical and oral surgery, including veterinary surgery, is shown. Laser-based surgical procedures may utilize any of a number of laser types, for example, a diode laser, an ER: YAG laser, a CO₂ laser, and the like, in combination with a non-reflective system. Advantageously, the system may be manufactured from materials that minimize the effect of heat from the laser on the instrument, as well as the patient. The use of metal functional elements in combination with a laser device suffers from many problems that include, for the purpose of illustration rather than limitation: transmitting the laser beam and potentially damaging underlying tissue, reflecting or scattering the laser beam, absorbing energy causing the functional element to overheat, emitting toxic or noxious vapors, and the like.

As described in greater detail below, the inventors have discovered that a nonreflective, cloudy, e.g. TPE, material minimizes the effect of heat on the instrument. More specifically, placing a soft, cloudy, clear material (e.g., TPE, polypropylene, polycarbonate) over a harder (e.g., plastic, polycarbonate, and the like) material.

In some implementations, the system 100 may include an elongate handle 10, having a proximal end 40 and a distal end 30, in combination with a functional element 20 that is operatively (e.g., fixedly or removably) coupled to the distal end 30 of the elongate handle 10. In some variations, the (e.g., plastic, TPE, and so forth) elongate handle 10 is ergonomically designed to fit comfortable in the hand of the user, further including a plurality of protrusions 15 that are structured and arranged on the elongate handle 10 to promote a firm grip on the system 100. To prevent slippage that may occur due to saliva or water used in the procedure, texture may be included on the functional element 20 to minimize slippage or movement of the patient's tongue during the procedure and/or on the proximal end 40 of the elongate handle 10 to minimize slippage of the user's grip.

As shown in FIGS. 1C and 1D, in a first embodiment, the functional element 20 may include an (e.g., planar) upper surface 22 having an arcuate perimeter and a (e.g., planar) lower surface 24 having an arcuate perimeter. Preferably, the arcuate perimeters of the upper surface 22 and the lower surface 24 are the same or substantially the same in dimension and shape. As shown in FIGS. 1B-1D, the width of the functional element 20 increases from a proximal end to a distal end. A slotted, planar or flat upper surface enables the practitioner to control the position of the tongue better, whereas a curved surface enables the capture tongue to roll off.

Advantageously, a convex sidewall 26 is more comfortable and may extend from the upper surface 22 to the lower surface 24, about the outer peripheral edge of the upper 22 and lower surfaces 24. In some applications, the first embodiment of the functional element 20 may include an opening (e.g., an indent or a slot 25) formed into the distal end of the functional element 20 and a connector 28 disposed at the proximal end of the functional element 20 for (e.g., fixedly or removably) attaching the functional element 20 to the elongate handle 10. In some variations, the indent or slot 25 at the distal end of the functional element 20 may be formed to include a pair of opposing walls 21, 23 that, like the convex sidewalls 26 that extend from the upper surface 22 to the lower surface 24, may also be convex. In some implementations, the length of the indent or slot 25 from the distal tip of the functional element 20 may range between about 0.430 and about 0.470 inches. Representative distances between the opposing sidewalls 21, 23 of the indent or slot 25 may range between about 0.60 and about 0.082 inches. The range for the indent or slot 25 width is limited because, during the procedure, the frenum may fold or bunch up in the slot 25 which may result in an uneven laser. The system 100 is designed to stretch and flatten the frenum for the cut.

Optionally, a hole 27 having an outer dimension (e.g., diameter) that exceeds the distance between the opposing sidewalls 21, 23 of the indent or slot 25 may be formed at the end of the indent or slot 25. Typical outer dimensions (e.g., diameters) for the hole 27 may range between about 0.120 and about 0.150 inches. The indent or slot 25 has a radius at the end so that the frenum is not jammed or caught for the infant. For adult use, the leading edge of the frenum tends to have a greater thickness; so, the hole 27 is captured and held allowing room for the extra thickness.

As shown in FIGS. 1E-1H, in a second embodiment, the functional element 50 that is fixedly attached to or removably attachable to the elongate handle 10 may include an (e.g., planar) upper surface 52 having an arcuate perimeter and a (e.g., planar) lower surface 54 having an arcuate perimeter. Preferably, the arcuate perimeters of the upper surface 52 and the lower surface 54 are the same or substantially the same in dimension and shape. As shown in FIGS. 1E and 1H, the width of the functional element 50 increases from a proximal end to a distal end.

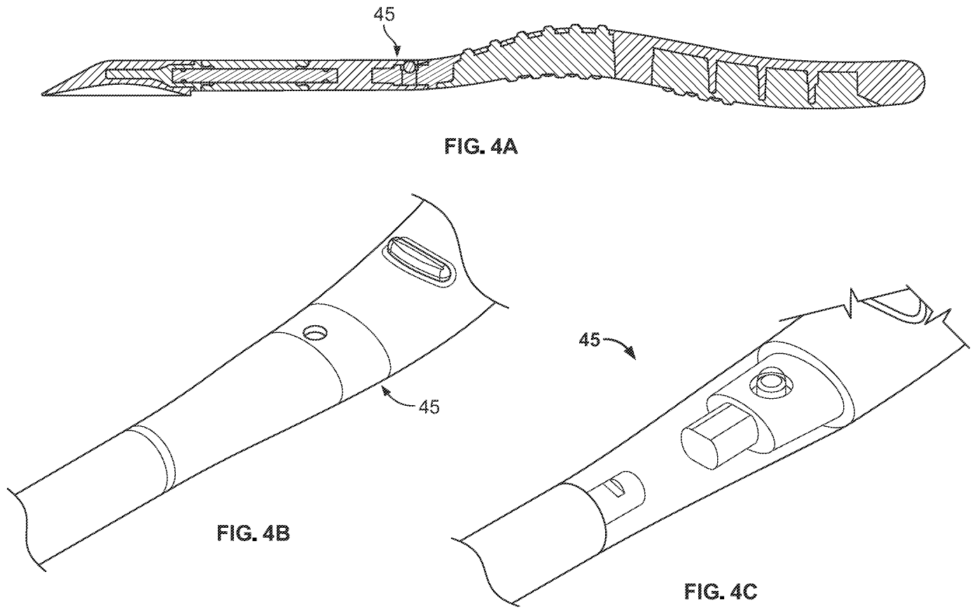

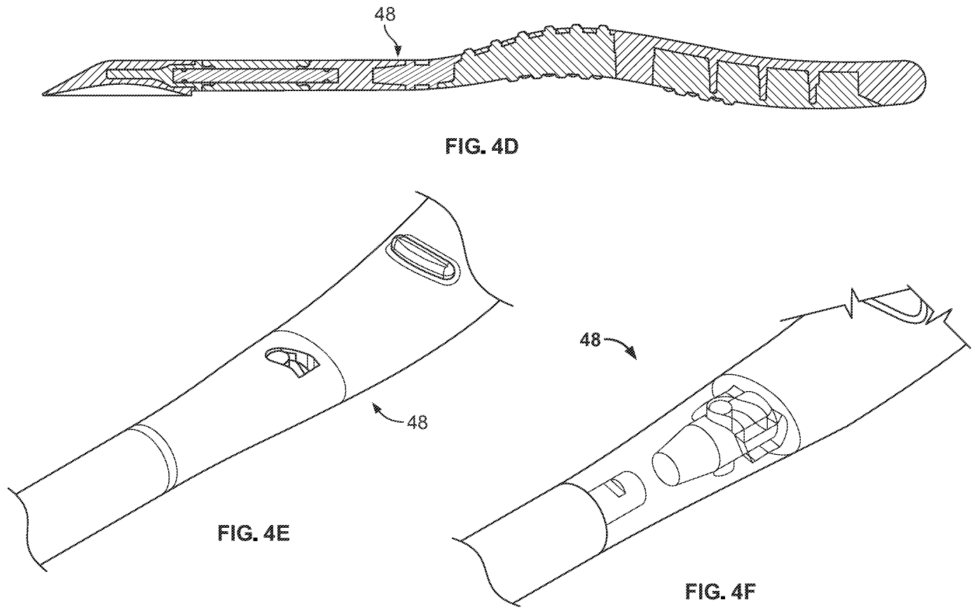

Advantageously, a convex sidewall 56 may extend from the upper surface 52 to the lower surface 54, about the outer peripheral edge of the upper 52 and lower surfaces 54. In some applications, the second embodiment of the functional element 50 may include an opening (e.g., an indent or a slot 55) formed into a distal end of the functional element 50 and a connector 58 disposed at the proximal end of the functional element 50 for (e.g., fixedly or removably) attaching the functional element 50 to the elongate handle 10. For the purpose of illustration rather than limitation, exemplary connectors 58 may include a friction, tight interference fit, or snap on connecting functional element; a snap on functional element with a ball detent 45 (FIG. 4A); a quarter-turn twist locking functional element 48 (FIG. 4B); and the like.

In some variations, the indent or slot 55 at the distal end of the functional element 50 may be formed to include a pair of opposing walls 51, 53. Advantageously, the opposing walls 51, 53 may be chamfered 59. Chamfering provides for greater comfort, especially when the system 100 captures the frenum and is then lifted up to hold and capture the patient's tongue. As the tongue is lifted, the frenum is stretched. If the instrument has a sharp edge (like the metal instruments) it can cut the tongue and cause bleeding.

In some implementations, the length of the indent or slot 55 from the distal tip of the functional element 50 may range between about 0.430 and about 0.470 inches. Representative distances between the opposing walls 51, 53 of the indent or slot 55 may range between about 0.60 and about 0.082 inches. The slot opening facilitates capturing of the frenum. If the lead in is longer, the system 100 would increase in length or the capture area in the indent or slot 55 would not be as tight.

Optionally, a hole 57 having an outer dimension (e.g., diameter) that exceeds the distance between the opposing walls 51, 53 of the indent or slot 55 may be formed at the distal end of the indent or slot 55. Typical outer dimensions (e.g., diameters) for the hole 57 may range between about 0.120 and about 0.150 inches.

As shown in FIGS. 1I-1K, in a third embodiment, the functional element 60 may include an (e.g., planar) upper surface 62 having an arcuate perimeter and a (e.g., planar) lower surface 64 having an arcuate perimeter. Preferably, the arcuate perimeters of the upper surface 62 and the lower surface 64 are the same or substantially the same in dimension and shape. As shown in FIGS. 1I and 1J, the width of the functional element 60 increases from a proximal end to a distal end.

A convex sidewall 66 may extend from the upper surface 62 to the lower surface 64, about the outer peripheral edge of the upper 62 and lower surfaces 64. In some applications, the third embodiment of the functional element 60 may include an opening (e.g., an indent or a slot 65) formed into a distal end of the functional element 60 and a connector 68 disposed at the proximal end of the functional element 60 for (e.g., fixedly or removably) attaching the functional element 60 to the elongate handle 10. For the purpose of illustration rather than limitation, exemplary connectors 68 may include a friction, tight interference fit, or snap on connecting functional element; a snap on functional element with a ball detent 45 (FIG. 4A); a quarter-turn twist locking functional element 48 (FIG. 4B); and the like.

In some variations, the indent or slot 65 may be formed in the functional element 60 to include a pair of opposing walls 61, 63 that, like the convex sidewalls 66 that extend from the upper surface 62 to the lower surface 64, may also be convex. Due to the draft angle of a molded part the opposing walls 61, 63 may never be truly planar (i.e., parallel). Indeed, the draft angle causes a slight chamfer and then has a convex shape and then the larger chamfer. Advantageously, the opposing walls 61, 63 may be chamfered 69. In some implementations, the length of the indent or slot 65 from the proximal tip of the functional element 60 may range between about 0.430 and about 0.470 inches. Representative distances between the opposing walls 61, 63 of the indent or slot 65 may range between about 0.60 and about 0.082 inches.

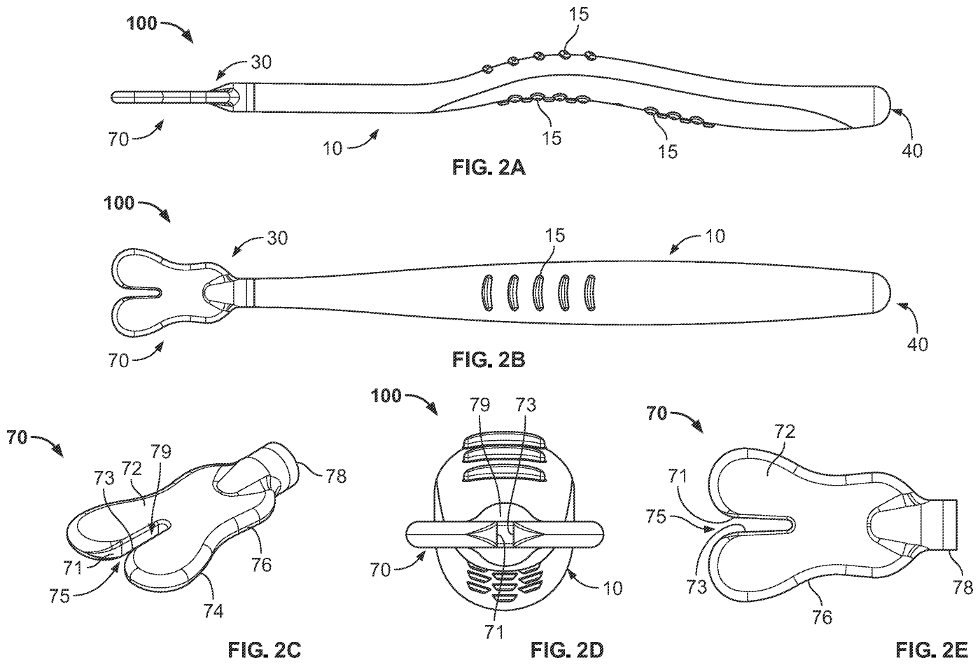

Referring to FIGS. 2A and 2B, a second embodiment of a system 100 for (e.g., laser-based) medical and oral surgery, including veterinary surgery, is shown. In some implementations, the system 100 may include an elongate handle 10, having a proximal end 40 and a distal end 30, in combination with a functional element 70 that is operatively (e.g., fixedly or removably) coupled to the distal end 30 of the elongate handle 10. In some variations, the (e.g., plastic, TPE, and so forth) elongate handle 10 is ergonomically designed to fit comfortable in the hand of the user, further including a plurality of protrusions 15 that are structured and arranged on the elongate handle 10 to promote a firm grip on the system 100. To prevent slippage that may occur due to saliva or water used in the procedure, texture may be included on the functional element 70 to minimize slippage or movement of the patient's tongue during the procedure and/or on the proximal end 40 of the elongate handle 10 to minimize slippage of the user's grip.

As shown in FIGS. 2C-2E, the functional element 70 may include an (e.g., planar) upper surface 72 having an arcuate perimeter and a (e.g., planar) lower surface 74 having an arcuate perimeter. Preferably, the arcuate perimeters of the upper surface 72 and the lower surface 74 are the same or substantially the same in dimension and shape. As shown in FIGS. 2B, 2C, and 2E, the width of the functional element 70 increases from a proximal end to a distal end.

Advantageously, a convex sidewall 76 may extend from the upper surface 72 to the lower surface 74, about the outer peripheral edge of the upper 72 and lower surfaces 74. Convex sidewalls 76 provide greater comfort than planar sidewalls as, typically, anesthesia is not used during most of these procedures. In some applications, the functional element 70 may include an opening (e.g., an indent or a slot 75) formed into a distal end of the functional element 70 and a connector 78 disposed at the proximal end of the functional element 70 for (e.g., fixedly or removably) attaching the functional element 70 to the elongate handle 10. For the purpose of illustration rather than limitation, exemplary connectors 78 may include a friction, tight interference fit, or snap on connecting functional element; a snap on functional element with a ball detent 45 (FIG. 4A); a quarter-turn twist locking functional element 48 (FIG. 4B); and the like.

In some variations, the indent or slot 75 may be formed in the functional element 70 to include a pair of opposing walls 71, 73. In some implementations, the length of the indent or slot 75 from the distal tip of the functional element 70 may range between about 0.430 and about 0.470 inches. Representative distances between the opposing walls 71, 73 of the indent or slot 75 may range between about 0.60 and about 0.082 inches. Advantageously, the opposing walls 71, 73 may be chamfered 79.

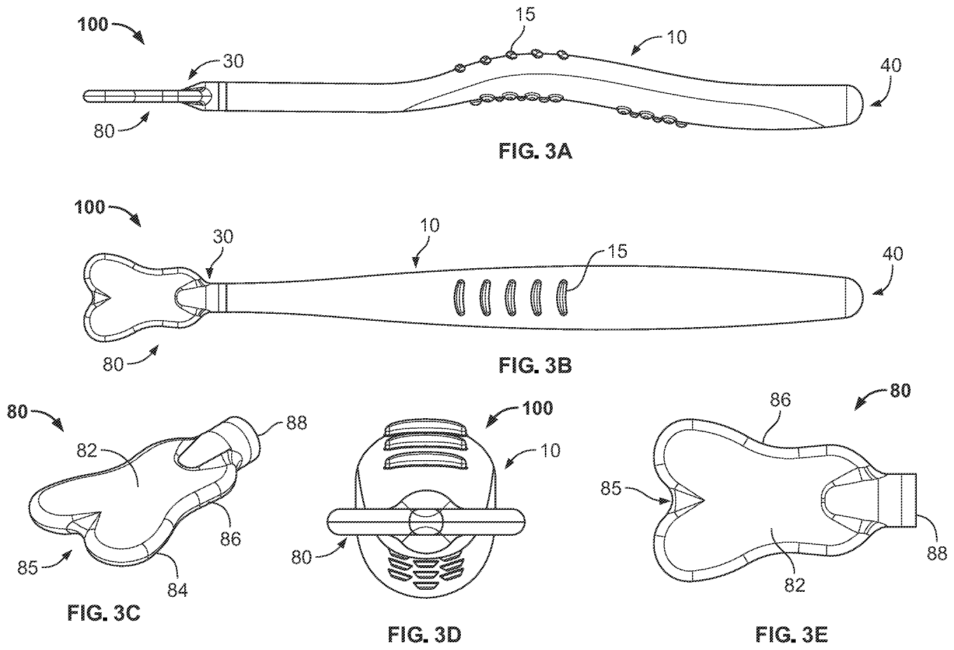

Referring to FIGS. 3A and 3B, a third embodiment of a system 100 for (e.g., laser-based) medical and oral surgery, including veterinary surgery, is shown. In some implementations, the system 100 may include an elongate handle 10, having a proximal end 40 and a distal end 30, in combination with a functional element 80 that is operatively (e.g., fixedly or removably) coupled to the distal end 30 of the elongate handle 10. In some variations, the (e.g., plastic, TPE, and so forth) elongate handle 10 is ergonomically designed to fit comfortable in the hand of the user, further including a plurality of protrusions 15 that are structured and arranged on the elongate handle 10 to promote a firm grip on the system 100. To prevent slippage that may occur due to saliva or water used in the procedure, texture may be included on the functional element 80 to minimize slippage or movement of the patient's tongue during the procedure and/or on the proximal end 40 of the elongate handle 10 to minimize slippage of the user's grip.

As shown in FIGS. 3C-3E, the functional element 80 may include an (e.g., planar) upper surface 82 having an arcuate perimeter and a (e.g., planar) lower surface 84 having an arcuate perimeter. Preferably, the arcuate perimeters of the upper surface 82 and the lower surface 84 are the same or substantially the same in dimension and shape. As shown in FIGS. 3B, 3C, and 3E, the width of the functional element 80 increases from a proximal end to a distal end.

Advantageously, a convex sidewall 86 may extend from the upper surface 82 to the lower surface 84, about the outer peripheral edge of the upper 82 and lower surfaces 84. In some applications, the functional element 80 may include a (e.g., heart-shaped, radiused) indent 85 formed into a distal end of the functional element 80 and a connector 88 disposed at the proximal end of the functional element 80 for (e.g., fixedly or removably) attaching the functional element 80 to the elongate handle 10. For the purpose of illustration rather than limitation, exemplary connectors 88 may include a friction, tight interference fit, or snap on connecting functional element; a snap on functional element with a ball detent 45 (FIG. 4A); a quarter-turn twist locking functional element 48 (FIG. 4B); and the like.

In some variations, the (e.g., heart-shaped, radiused) indent tongue 85 allows the parent to exercise the infant's tongue to prevent reattachment of the wound without contacting the wound. The heart-shaped area of the tip keeps the instrument clear of the wound.

Universal Handle and Disposable Tips

Including a friction, tight interference fit, or snap on connecting device; a snap on device with a ball detent 45 (FIG. 4A); a quarter-turn twist locking device 48 (FIG. 4B); and the like to the elongate handle 10 promotes the use of an autoclavable universal handle that is structured and arranged to be connectable to a plurality of functional elements 20, 40, 50, 60, 70, 80 so as to be reusable. Advantageously, users may purchase a single autoclavable universal handle and a plurality of different functional elements 20, 40, 50, 60, 70, 80, as needed, each of which is removably attachable to the universal handle.

Buccal Tip on Elongate Handle

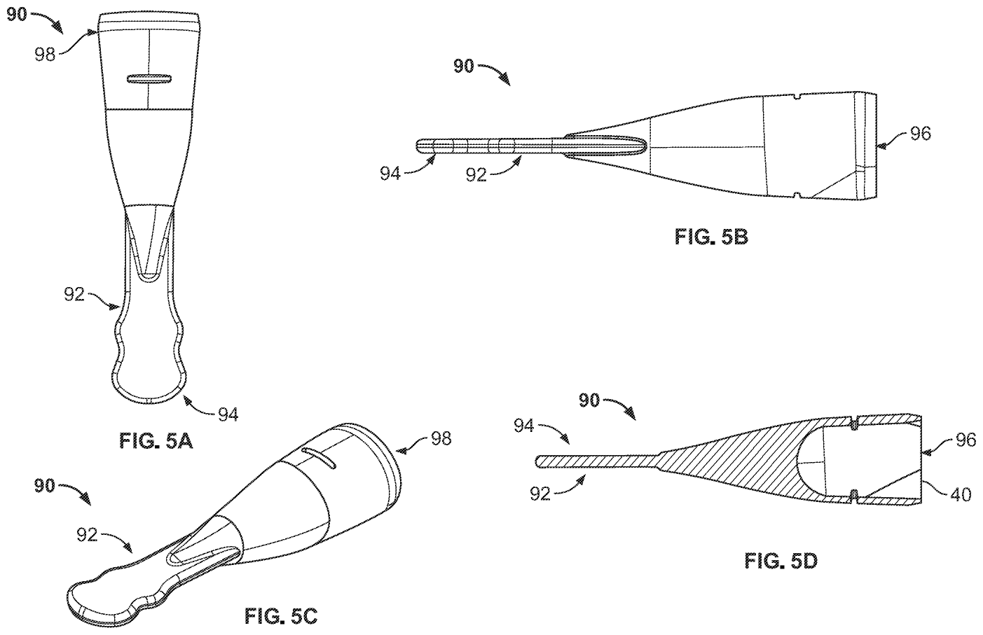

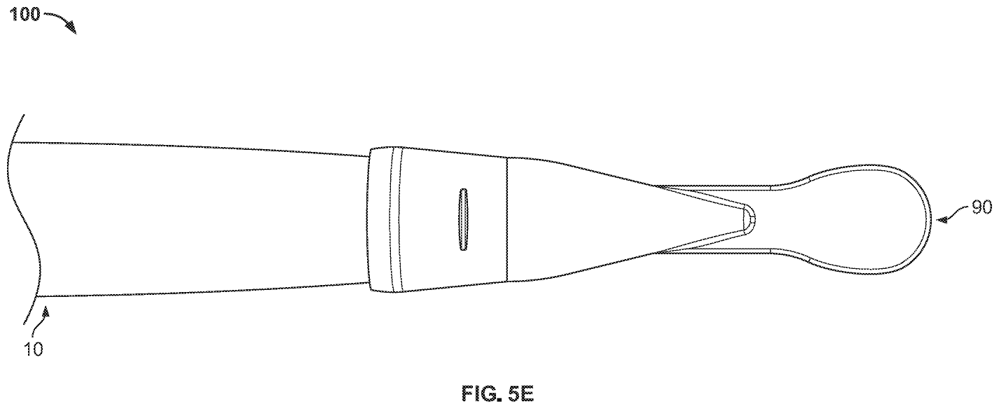

In some embodiments of the system 100, as shown in FIGS. 5A-5D, the proximal end 40 of the elongate handle 10 may be structured and arranged to accommodate a second (e.g., buccal tip) functional element 90 that may be configured to perform a buccal tie release procedure immediately after a frenectomy, especially for such procedures for small children. Advantageously, the user is able use their hand to reverse or rotate the handle 10 approximately 180 degrees without having to put down the system 100 and re-grab it and without having to lose the patient position. Although the second functional element 90 will be described for a (e.g., disposable) buccal tip embodiment, those of ordinary skill in the art can appreciate that the second functional element may be a mirror, a tissue retraction element, a tongue guide a laser blocking element, a brush, a hook, a block, and the like.

As shown in FIGS. 5B-5D, the buccal tip functional element 90 may include a rounded, relatively flat or slightly convex tip portion 92 at a distal end 94 of the functional element 90 and an opening 96 at a proximal end 98 of the functional element 90. Preferably, the opening 96 of the buccal tip functional element 90 is dimensioned to provide a tight, interference fit with the proximal end 40 of the elongate handle 10, such as occurs with a pen cap. Optionally, the proximal end 40 of the elongate handle 10 may be configured to include an indent feature that is structured and adapted to mate with and to receive a corresponding projection feature incorporated in the opening 96 at the proximal end 98 of the buccal tip functional element 90. Alternatively, the proximal end 40 of the elongate handle 10 may be configured to include a projection feature that is structured and adapted to mate with a corresponding indent feature incorporated in the opening 96 at the proximal end 98 of the buccal tip functional element 90.

Tissue Retraction Element with Soft Integral Covering

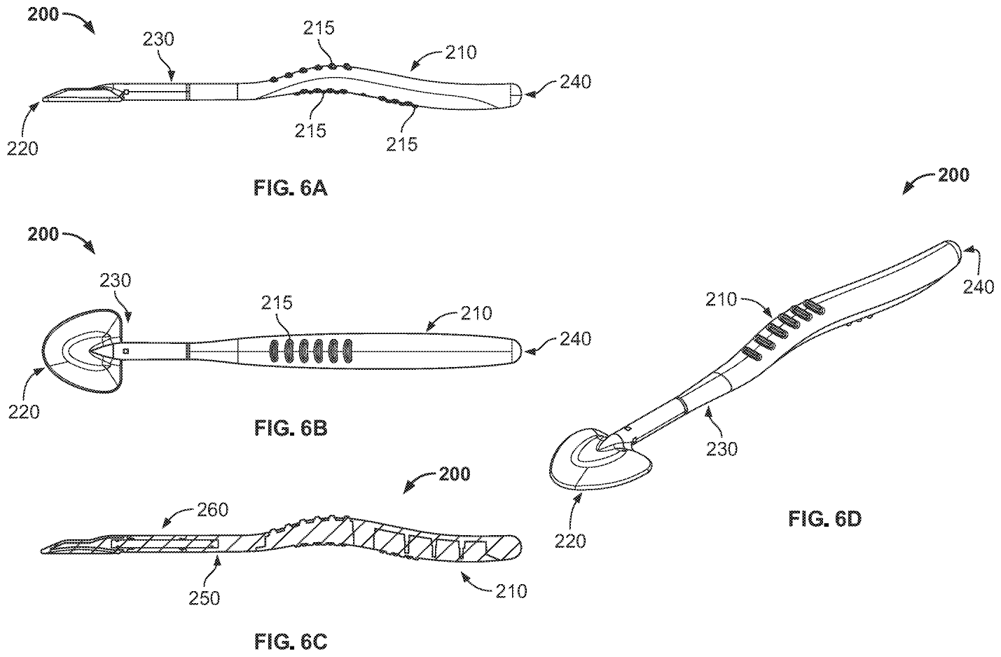

Referring to FIGS. 6A-6D, an embodiment of a tissue retraction system 200 having a soft integral covering or coating is shown. In some implementations, the tissue retraction system 200 includes a proximal end 240 and a distal end 230, in combination with a tissue retraction functional element 220 that is operatively (e.g., fixedly) coupled to the distal end 230 of the elongate handle 210. In some variations, the rigid or semi-rigid (e.g., plastic, polycarbonate, and so forth) elongate handle 210 is ergonomically designed to fit comfortable in the hand of the user. In some variations, the (e.g., plastic, polycarbonate, and so forth) elongate handle 210 is ergonomically designed to fit comfortable in the hand of the user, further including a plurality of protrusions and softer portions 215 that are structured and arranged on the elongate handle 210 to promote a firm grip on the system 200. To prevent slippage that may occur due to saliva or water used in the procedure, texture may be included on the tissue retraction functional element 220 to minimize slippage or movement of the patient's tongue or cheeks during the procedure and/or on the proximal end 240 of the elongate handle 210 to minimize slippage of the user's grip.

In some applications, the substantially rigid or semi-rigid (e.g. polycarbonate) elongate handle 210 of the retraction system 200 includes a selectively bendable (e.g., gooseneck) neck portion at or proximate the distal end 230. In some variations, the distal end 230 is made bendable by incorporating a soft, malleable element, pin, or rod 250 in the neck portion of the distal end 230 of the elongate handle 210. Exemplary metals for use in the neck portion of the distal end 230 of the elongate handle 210 include aluminum 1100, fully annealed copper C1100, annealed stainless steel 316, and the like. Although the invention is described as including a soft, malleable element, pin, or rod 250, those of ordinary skill in the art can appreciate that, in some implementations of the system 200, movement of the tissue retraction functional element 220 may not be desirable. Hence, the element, pin, or rod 250 disposed in the neck portion of the distal end 230 of the elongate handle 210 does not have to be soft or malleable and can be, instead, rigid (e.g., stainless steel) to achieve extended life cycles.

Because of the discomfort and potential damage that may result if portions of the substantially rigid or semi-rigid (e.g., polycarbonate) elongate handle 210 impact or come into contact with the patient's oral tissue, gingiva, teeth, and so forth, an integral soft (e.g., TPE and the like) covering or coating 260 may be formed about the substrates of the elongate handle 210 and the tissue retraction functional element 220. More particularly, soft, flexible polymers may be chemically bonded to the rigid polymer substrates of the elongate handle 210 and the tissue retraction functional element 220. The flexible polymers provides a soft, comfortable surface that is less likely to damage sensitive oral tissue during procedures. When TPE is used as the soft polymer, a durometer range between 30 and 40 Shore A is preferred; however, for intubation and/or gynecological applications, a durometer range between 10 and 25 Shore A is preferred. Advantageously, a TPE such as Softlink 140A may be autoclavable in excess of 60 cycles.

Mirror Functional Element with Soft Integral Covering

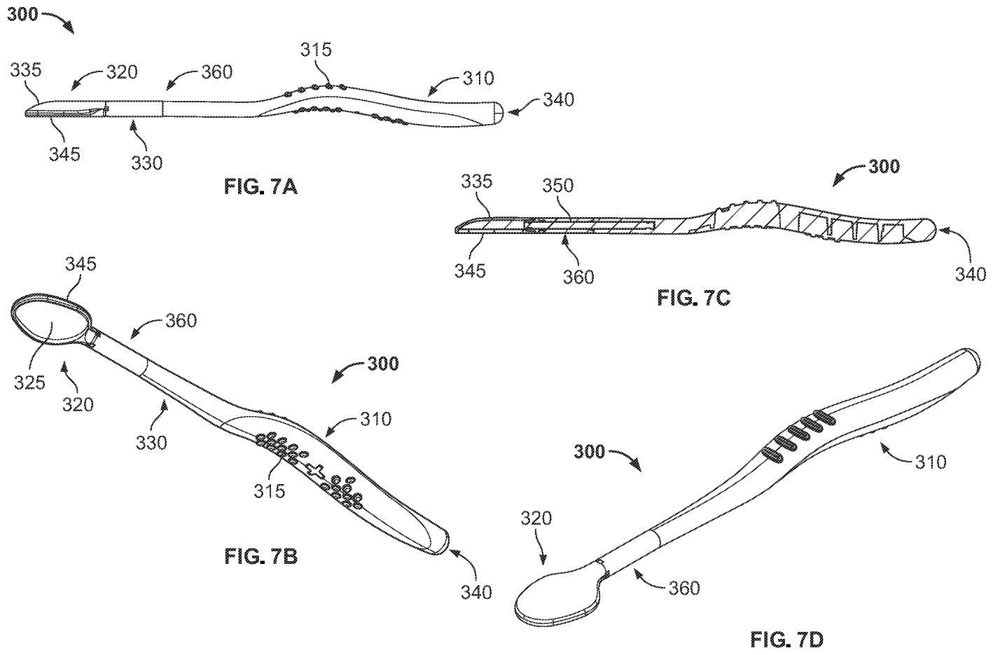

Referring to FIGS. 7A-7D, an embodiment of a mirror system 300 having a soft integral covering or coating is shown. In some implementations, the mirror system 300 includes a proximal end 340 and a distal end 330, in combination with a mirror functional element 320 that is operatively (e.g., fixedly) coupled to the distal end 330 of the elongate handle 310. More specifically, the mirror functional element 320 includes a reflective glass portion 325 (i.e., the mirror) that is mounted within a frame or substrate portion 335 that includes a bumper portion 345 adjacent to the peripheral surface of the reflective glass portion 325. Advantageously, the bumper portion 345 is configured to retain as well as provide a seal against the reflective glass portion 325. Those of ordinary skill in the art can appreciate that if moisture gets behind the reflective glass portion 325, fogging may result and/or the coating may be affected.

In some variations, the rigid or semi-rigid (e.g., plastic, polycarbonate, and so forth) elongate handle 310 is ergonomically designed to fit comfortable in the hand of the user. In some variations, the (e.g., plastic, polycarbonate, and so forth) elongate handle 310 is ergonomically designed to fit comfortable in the hand of the user, further including a plurality of protrusions and softer portions 315 that are structured and arranged on the elongate handle 310 to promote a firm grip on the system 300. To prevent slippage that may occur due to saliva or water used in the procedure, texture may be included on the mirror functional element 320 to minimize slippage or movement of the patient's tongue or cheeks during the procedure and/or on the proximal end 340 of the elongate handle 310 to minimize slippage of the user's grip.

In some applications, the substantially rigid or semi-rigid (e.g. polycarbonate) elongate handle 310 of the retraction system 300 includes a selectively bendable (e.g., gooseneck) neck portion at or proximate the distal end 330. In some variations, the distal end 330 is made bendable by incorporating a soft, malleable element, pin, or rod 350 in the neck portion of the distal end 330 of the elongate handle 310. Exemplary metals for use in the neck portion of the distal end 330 of the elongate handle 310 include aluminum 1100, fully annealed copper C1100, annealed stainless steel 316, and the like. Although the invention is described as including a soft, malleable element, pin, or rod 350, those of ordinary skill in the art can appreciate that, in some implementations of the system 300, movement of the mirror functional element 320 may not be desirable. Hence, the element, pin, or rod 350 disposed in the neck portion of the distal end 330 of the elongate handle 310 does not have to be soft or malleable and can be, instead, rigid (e.g., stainless steel) to achieve extended life cycles.

Because of the discomfort and potential damage that may result if portions of the substantially rigid or semi-rigid (e.g., polycarbonate) elongate handle 310 impact or come into contact with the patient's oral tissue, gingiva, teeth, and so forth, an integral soft (e.g., TPE and the like) covering or coating 360 may be formed about the elongate handle 310, the frame or substrate portion 335, and the bumper portion 345 adjacent to the peripheral surface of the reflective glass portion 325. Preferably, the entire functional element 320, including the bendable neck, may be covered by the integral soft (e.g., TPE and the like) covering or coating 360 to provide comfort and protection for the patient.

More particularly, soft, flexible polymers may be chemically bonded to the rigid polymer substrates of the elongate handle 310 and the frame or substrate portion 335 and the bumper portion 345 adjacent to the peripheral surface of the reflective glass portion 325 of the mirror functional element 320. The flexible polymers provides a soft, comfortable surface that is less likely to damage sensitive oral tissue during procedures. When TPE is used as the soft polymer, a durometer range between 30 and 40 Shore A is preferred; however, for intubation and/or gynecological applications, a durometer range between 10 and 25 Shore A is preferred. Advantageously, a TPE such as Softlink 140A may be autoclavable in excess of 60 cycles.

Kit

Advantageously, any of the systems 100, 200, 300 that include one or more of the plurality of functional elements 10, 50, 60, 70, 80, 90, 220, 320 may be included with one or more of any other system 100, 200, 300 or functional element 10, 50, 60, 70, 80, 90, 220, 320 and/or with other oral hygiene functional elements (e.g., a tooth brush, a flosser, a gingiva massager, a scaling functional element, and the like) in a kit. For example, one kit may include a tooth brush and a retractor system 200. The retractor in such a combination improves access and visibility and the tooth brush, having greater access and visibility, improves hygiene.

Laser Compliant Functional Elements

In various embodiments, the present invention also relates to a functional element for use with oral or medical surgery that is more laser compliant than conventional functional elements. As used herein, laser compliant means that the functional element exhibits a safer and/or favorable responses to interaction with a laser than conventional functional elements. For example, the functional element may exhibit a lower increase in temperature, lower reflectivity of laser radiation, and/or less smoking or charring when interacting with laser energy than conventional functional elements. In some embodiments, the functional element is particularly configured to have superior performance/interaction with a particular type of laser (e.g., Er: YAG, diode-based, or CO₂) and/or to have superior performance/interaction with a particular laser wavelength (e.g., in a range from 2-4 μm, in a range from 9-11 μm).

In some embodiments, superior laser compatibility can be a result of the type, color, and/or translucency of the material from which the laser interacting portion of the functional element is formed. Through testing, Applicant has discovered that a solid white TPE component can interact with a diode laser with no adverse effect (e.g., no sparking, smoking, etc.). Applicant also discovered that a cloudy/clear TPE tip can interact with an Er: YAG laser with no adverse effect (e.g., no sparking, smoking, etc.). In some embodiments, the use or water (or other coolant) in conjunction with delivery of the laser energy can affect the types of materials that are laser compliant. For example, Applicant discovered that a cloudy/clear TPE material will spark when interacting with a CO₂ laser without water, but does not spark or have other adverse effect when interacting with a CO₂ laser with water. Applicant has discovered that a cloudy/clear TPE material can interact with an Er: YAG laser (e.g., manufactured by Fotona™) or a diode-based laser (e.g., manufactured by Biolase™) without generating any smoke or spark, regardless of whether water is present. Furthermore, Applicant has discovered that a cloudy/clear TPE material can interact with a CO₂ laser (e.g., manufactured by Convergent Dental™) without generating any smoke or spark, if water is present.

It should be understood that alternative embodiments and/or materials used in the construction of embodiments, or alternative embodiments, are applicable to all other embodiments described herein. Furthermore, the invention may be embodied in other specific forms without departing from the spirit or essential characteristics thereof. The foregoing embodiments, therefore, are to be considered in all respects illustrative rather than limiting the invention described herein. Scope of the invention is thus indicated by the appended claims, rather than by the foregoing description, and all changes that come within the meaning and range of equivalency of the claims are intended to be embraced therein.

Bite Block/Patient Airway Protection Functional Element

In any embodiment, the present invention relates to a bite block/patient airway protection functional element that is structured and arranged to include a rigid material that is overmolded with a soft covering (e.g., TPE). In some implementation, the bite block may also include metal (e.g., spring-like) feature that are configured to enable the functional element to expand in response to movement (e.g., grinding teeth) occurring when a patient is placed on a ventilator.

The soft covering on the bite block is adjustable and protects the patient's lips and teeth from damage and oral tissue and gingiva from becoming inflamed, while preventing the ventilator tube from becoming compromised. In some variations, the soft material used for the outer covering may also precent the build-up of bacteria.

The bite block may be used in numerous applications and for different reasons. For example, during dental procedures the bite block may be used to keep the mouth open to a degree that allows different dental functions. Bite blocks are widely used for special needs children that are not able to assist the dentist with mouth positions.

More importantly the bite block may be used to keep the patient's jaw/mouth open during prolonged intubation. During prolonged periods for surgery or coma, the airway tube must stay open and the teeth, tongue, lips and gums need to be protected from grinding, biting, and epileptic fits. The bite block will embody a similar rigid positioning function with soft and protective TPE covering surfaces to protect the tongue, gums, and lips. In some embodiments, the bite block may include ridged functional elements to allow for controlled movement of the jaw while protecting the soft tissue. Optionally, the bite block may include protection of the airway tube separately from protection of the teeth and soft tissue.

Having described herein illustrative embodiments of the present invention, persons of ordinary skill in the art will appreciate various other features and advantages of the invention apart from those specifically described above. It should therefore be understood that the foregoing is only illustrative of the principles of the invention, and that various modifications and additions can be made by those skilled in the art without departing from the spirit and scope of the invention. Accordingly, the appended claims shall not be limited by the particular features that have been shown and described but shall be construed also to cover any obvious modifications and equivalents thereof.

What is claimed is:

1. A system comprising:
an elongate handle comprising a proximal end and a distal end;
a functional element disposed at the distal end of the elongate handle; and
an integral, soft coating material formed about, so as to cover, the functional element and the distal end of the elongate handle.

2. The system of claim 1, wherein the elongate handle comprises a rigid portion and a soft portion for improving a user's grip of the elongate handle.

3. The system of claim 2, wherein the rigid portion comprises a polycarbonate and the soft portion comprises a thermoplastic elastomer.

4. The system of claim 1, wherein the functional element is disposable and selectively removable from the elongate handle.

5. The system of claim 4, wherein the functional element forms a tight, interference fit with the distal end of the elongate handle.

6. The system of claim 4, wherein the distal end of the elongate handle comprises at least one of a quarter-turn twist on element or a ball detent for attaching the functional element to the distal end of the elongate handle.

7. The system of claim 1, wherein the functional element comprises at least one of a mirror, a tissue retraction element, a laser blocking element, a brush, a hook, a block, and a buccal tip.

8. The system of claim 1, wherein the system further comprises a second functional element that is selectively attachable to and removably from the proximal end of the elongate handle.

9. The system of claim 8, wherein the second functional element is disposable.

10. The system of claim 8, wherein the second functional element comprises at least one of a mirror, a tissue retraction element, a tongue guide a laser blocking element, a brush, a hook, a block, and a buccal tip.

11. The system of claim 8, wherein the proximal end of the elongate handle comprises an indent feature that is structured and arranged to mate with a projection feature formed in the second functional element.

12. The system of claim 8, wherein the proximal end of the elongate handle comprises a projection feature that is structured and arranged to mate with an indent feature formed into the second functional element.

13. The system of claim 1 further comprising at least one of an element, pin, or rod disposed in a neck portion of the distal end of the elongate handle.

14. The system of claim 13, wherein the at least one of an element, pin, or rod is selected from the group consisting of stainless steel, aluminum 1100, fully annealed copper C1100, and stainless steel 316.

15. The system of claim 13, wherein the at least one of an element, pin, or rod is pliable so that neck portion is formable to provide a gooseneck appearance.

16. The system of claim 1, wherein the integral, soft covering is manufactured of a soft polymer selected from the group consisting of a thermoplastic elastomer, a thermoplastic elastomer having a durometer value between 10 and 25 Shore A, or a thermoplastic elastomer having a durometer value between 30 and 40 Shore A.

17. The system of claim 1, wherein the functional element comprises a mirror.

18. The system of claim 17, wherein the integral, soft coating material seals about a peripheral surface of the mirror.

19. The system of claim 17, wherein the integral, soft coating material forms a bumper about a peripheral surface of the mirror.

20. The system of claim 17, wherein the mirror is disposed within a substrate manufactured from at least one of plastic, a thermoplastic elastomer, and combinations thereof.

21. An oral hygiene kit comprising:
the system of claim 1, and
at least one oral hygiene functional element.

22. The kit of claim 21, wherein the at least one oral hygiene functional element is selected from the group consisting of a tooth brush, a flossing functional element, a gingiva stimulating functional element.
